(19) United States
(12) Patent Application Publication
(10) Pub. No.: US 2024/0091061 A1
(43) Pub. Date: Mar. 21, 2024

(54) PROBE DEVICE

(71) Applicant: Microport Visionpower Medtech (Shanghai) Co Ltd, Shanghai (CN)

(72) Inventors: Renquan YANG, Shanghai (CN); Qiou CHEN, Shanghai (CN); Jie ZHANG, Shanghai (CN); Zhaohua CHANG, Shanghai (CN)

(73) Assignee: Microport Visionpower Medtech (Shanghai) Co Ltd, Shanghai (CN)

(21) Appl. No.: 18/470,453

(22) Filed: Sep. 20, 2023

(30) Foreign Application Priority Data
Sep. 21, 2022 (CN) 202211152583.0

(51) Int. Cl. A61F 9/007

(52) U.S. Cl. CPC A61F 9/00736; A61B 2017/320064; A61B 2217/005

(57) ABSTRACT

The present invention provides a probe device comprising an inner cutting probe and an outer cutting probe. wherein the outer cutting probe has a first side cutting and sucking port in communication with a sliding channel thereof, and the inner probe is slidable within the sliding channel of the outer cutting probe and shaped to have a first end blade edge, a second side port with a second side blade edge and a third side blade edge opposite to the second side blade edge in a cross-sectional view thereof. When the inner cutting probe is slidable within the sliding channel of the outer cutting probe in the forward and backward direction of one cutting cycle, three cuttings are achieved in the forward and backward direction of one cutting cycle, thereby improving the cutting efficiency of the operation.

### FIELD OF TECHNOLOGY

The present invention relates to the technical field of medical devices, in particular to a probe device.

### BACKGROUND

In some anterior segment cataract surgeries, a posterior capsule bearing the lens ruptures and the vitreous body behind the capsule overflows with the rupture. The vitreous body is a transparent viscoelastic filamentary material that is attached to the retina. If the overflown vitreous body is not removed in time, the retina will be pulled due to suction disturbance, resulting in blindness.

In more complex posterior segment surgeries such as vitreous body hemorrhage, retinal detachment, macular hole, etc. A cutting probe for the vitreous body becomes an essential key surgical instrument. However, the current cutting probe for the vitreous body has low cutting efficiency, which affects the process of surgery.

### SUMMARY

It is an object of the present invention to provide a probe that solves the problem of poor vitrectomy efficiency and improves the cutting efficiency of surgery.

In order to achieve the above object, an embodiment of the present invention provides a probe device comprising:
an inner cutting probe;
an outer cutting probe;
wherein the outer cutting probe has a first tube shape with a sliding channel along a first tube longitudinal axis thereof, and the outer cutting probe has an outer side wall with a first side cutting and sucking port in communication with the sliding channel, wherein the first side cutting and sucking port is configured to suck a vitreous body;
the inner cutting probe with a second tube shape is configured to penetrate through the sliding channel of the outer cutting probe and be slidable with respect to the outer cutting probe, the inner cutting probe having a suction channel along a second tube longitudinal axis thereof, the inner probe has a front end with an end surface defining a first end blade edge in communication with the suction channel of the inner cutting probe, an outer side wall of the inner probe is shaped to have a second side port with a top port edge, a bottom port edge, a second side blade edge and a third side blade edge opposite to the second side blade edge in a cross-sectional view thereof, a length of the top port edge is smaller than a length of the bottom port edge, the second side blade edge is located closer to the first end blade edge than the third side blade edge;
wherein the inner cutting probe is configured to have a left wall edge and a right wall edge, the top port edge is defined between the left wall edge and the right wall edge, a first cutting blade tip is formed, with a first cutting blade tip angle between the first end blade edge and the left wall edge ranging from 40 degrees to 60 degrees; a second cutting blade tip is formed, with a second cutting blade tip angle between the third side blade edge and the right wall edge ranging from 40 degrees to 60 degrees; and a third cutting blade tip is formed, with a third cutting blade tip angle between the second side blade edge and the left wall edge ranging from 40 degrees and 60 degrees;
when the inner cutting probe is slidable within the sliding channel of the outer cutting probe in a forward direction, the first end blade edge is to perform a first cutting by the first cutting blade tip piercing into the vitreous body of the outer cutting probe along the forward direction, and the inner cutting probe continues to move forwardly such that the third side blade edge is to perform a second cutting by the second cutting blade tip piercing into the vitreous body of the outer cutting probe along the forward direction;
when the inner cutting probe is slidable within the sliding channel of the outer cutting probe in a backward direction, merely the second side blade edge is to perform a third cutting by the third cutting blade tip piercing into the vitreous body of the outer cutting probe along the backward direction.

Alternatively, wherein an opening is formed between second side blade edge and the third the third side blade edge

Alternatively, wherein the first end blade edge is formed on a peripheral edge of a front port of an expanded cylinder, an outer diameter of the expanded cylinder gradually increases in a forward direction from the a right portion of the inner cutting probe towards a left portion of the inner cutting probe, and an outer side wall of the expanded cylinder at least partially engages with the inner side wall of the sliding channel of the outer cutting probe.

Alternatively, wherein one or more cutting grooves are provided on a peripheral edge of the front port of the expanded cylinder and configured to extend from the left portion of the inner cutting probe towards the right portion of the inner cutting probe, and the outer diameter of a front end of the expanded cylinder with one or more cutting grooves is greater than or equal to the inner diameter of a corresponding end of the sliding channel of the outer cutting probe; and
the expanded cylinder can penetrate through the sliding channel when the expanded cylinder is deformed by applying a force thereto.

The advantageous effect of the present invention is that by providing a first end blade edge, a second side blade edge and a third side cutting edge on the inner probe, when the inner cutting probe is slidable within the sliding channel of the outer cutting probe in the forward and backward direction of one cutting cycle, three times of cutting can be achieved with the three shears formed in one cutting cycle, which greatly improves the cutting efficiency of surgery.

### BRIEF DESCRIPTION OF THE DRAWINGS

FIG. 1 is a schematic view showing a structure of a probe device according to an embodiment of the present invention;

FIG. 2 is a section view of FIG. 1;

FIG. 3 is a schematic view showing a structure of a probe device according to another embodiment of the present invention;

FIG. 4 is a section view of FIG. 3;

FIG. 5 is a schematic view showing a structure of a probe device according to yet another embodiment of the present invention;

FIG. 6 is a section view of FIG. 5;

FIG. 7 is a schematic view showing a structure of a probe device according to yet another embodiment of the present invention;

FIG. 8 is a cross-sectional view of FIG. 7.

### REFERENCE NUMERALS

inner cutting probe 100, suction channel 101, first end blade edge 102, second side blade edge 103, third side blade edge 104, opening 105, cutting groove 106; outer cutting probe 200, sliding channel 201, first side cutting and sucking port 202.

### DESCRIPTION OF THE EMBODIMENTS

In order that the objects, aspects, and advantages of the present invention will become more fully apparent, embodiments of the present invention will be described hereinafter with reference to the accompanying drawings. Based on the embodiments of the present invention, all other embodiments obtained by a person skilled in the art without inventive effort fall within the scope of the present invention. Unless otherwise mentioned, all technical and scientific terms used herein have the same meaning as commonly understood by one of ordinary skill in the art to which the invention belong. As used herein, the terms “comprises”, “comprising”, and the like are intended to mean that the presence of an element or item preceding the term encompasses the presence of the listed element or item following the term and equivalents thereof, but does not exclude other elements or items.

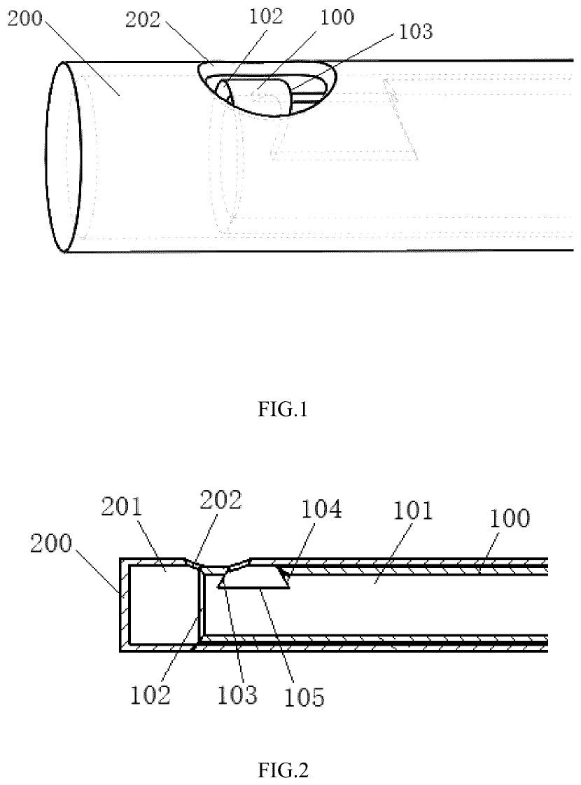

In view of the problems existing in the prior art, an embodiment of the present invention provides a probe device, as shown with reference to FIGS. 1 and 2, comprising an inner cutting probe or tube 100 and an outer cutting probe or tube 200, wherein the outer cutting probe 200 has a tube shape with a sliding channel 201 along a tube longitudinal axis thereof, and the outer cutting probe 200 has an outer sidewall with a first side cutting and sucking port 202 serving to suck the vitreous body or tissue, which will enter into the first side cutting and sucking port 202 of the outer cutting probe 200. The side cutting and sucking port 202 is in communicates with its sliding channel 201, and is located close to a front end of the outer cutting probe 200. The inner cutting probe 100 is configured to penetrate through the sliding channel 201 of the outer cutting probe 200 so that the inner cutting probe 100 can move within or be slidable with respect to the outer cutting probe 200. The inner cutting probe 100 also has a tube shape with a suction channel 101 along its tube longitudinal axis thereof, the inner cutting probe 100 has a front end with an end surface defining a first end blade edge 102 in communication with its suction channel 101, wherein the first end blade edge is inclined toward the inner of the inner cutting probe 100. An outer side wall of the inner cutting probe 100 is shaped to have a second side port with a top port edge (it is void), a bottom port edge, a second side blade edge 103 and a third side blade edge 104 in a cross-sectional view thereof, wherein a length of the top port edge is smaller than a length of the bottom port edge. The inner cutting probe 100 is thus shaped to have a left wall edge and a right wall edge, wherein the top port edge (it is void) is defined between the left wall edge and the right wall edge. A first cutting blade tip is formed, with a first cutting blade tip angle between the first end blade edge 102 and the left wall edge ranging from about 40 degrees to about 60 degrees. A second cutting blade tip is formed, with a second cutting blade tip angle between the third side blade edge 104 and the right wall edge ranging from about 40 degrees to about 60 degrees. A third cutting blade tip is formed, with a third cutting blade tip angle between the second side blade edge 103 and the left wall edge ranging from about 40 degrees to about 60 degrees. In a preferred embodiment, the first cutting blade tip angle ranges from 40 degrees to 60 degrees, the second cutting blade tip angle ranges from 40 degrees to 60 degrees, and the third cutting blade tip angle ranging from 40 degrees to 60 degrees. It should be noted that if the first end blade edge 102, the second side blade edge 103 or third side blade edge 104 is inclined and curved, the above-described blade tip angle is defined by a tangent line or edge of the first end blade edge 102, the second side blade edge 103 or third side blade edge 104. The side port of the inner cutting probe 100 is thus provided with the second side blade edge 103 and the third side blade edge 104 arranged opposite to the second side blade edge 103, which is close to the first end blade edge 102, and the second side blade edge 103 and the third side blade edge 104 are also in communication with the suction channel 101 of the inner cutting probe 100. Herein, the second side blade edge 103 is located closer to the first end blade edge 102 than the third side blade edge 104.

In a preferred embodiment, by providing the first end blade edge 102, the second side blade edge 103 and the third side blade edge 104 on the inner probe 100, three cuttings are achieved in a forward and backward movement in one cutting cycle, greatly improving the cutting efficiency of the surgery.

In implementations, when the inner cutting probe 100 is slidable or moves within the sliding channel 201 of the outer cutting probe 200 in a forward direction, the first end blade edge 102 is to firstly perform or achieve a first cutting by the first cutting blade tip piercing into the vitreous body of the outer cutting probe 200 along the forward direction, and the inner cutting probe 100 continues to move forwardly such that the third side cutting edge 104 is to secondly perform or achieve a second cutting by the second cutting blade tip piercing into the vitreous body of the outer cutting probe 200 along the forward direction. When the inner cutting probe 100 is slidable or moves within the sliding channel 201 in a backward direction, merely the second side blade edge 103 is to perform or achieve a third cutting by the third cutting blade tip piercing into the vitreous body of the outer cutting probe 200 along the backward direction.

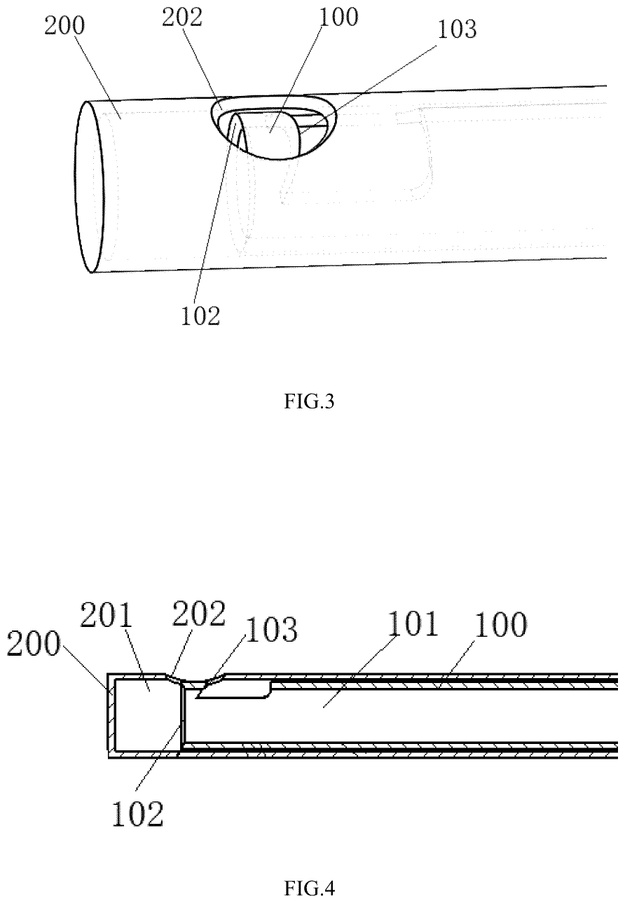

In some embodiments, as shown with reference to FIGS. 3 and 4, the inner probe 100 is merely provided with the first end blade edge 102 and the second side blade edge 103 with no third side blade edge 104, and the first end blade edge 102 and the second side blade edge 103 are shaped to have the corresponding cutting blade tips, which will easily break the vitreum fiber by the sharp cutting blade tip of the first end blade edge 102 or the second side blade edge 103.

Further, in an embodiment, cutting-edge angles of the first end blade edge 102, the second side blade edge 103, and the third side blade edge 104 104 each range from about 40 degrees to about 60 degrees.

In the embodiment, by setting the cutting-edge angle of the first end blade edge 102, the second side blade edge 103, or the third side blade edge 104 within a range of 40 degrees to 60 degrees, it is easier to cut the vitreum fiber or tissue, thereby reducing the traction on the retina during the operation, and at the same time, the cut vitreum fragment can flow more smoothly in the presence of the cutting-edge angle, thereby improving the efficiency of sucking vitreous body or tissue.

In some embodiments, each of the cutting-edge angles of the first end blade edge 102 and the second side blade edge 103 has an angle of 40 degrees. In other embodiments, each of the cutting-edge angles of the first end blade edge 102 and the second side blade edge 103 has an angle of 60 degrees, which is not enumerated here.

Alternatively, as shown with reference to FIG. 2, an opening 105 is provided between the second side blade edge 103 and the third side blade edge 104, and first end blade edge 102, the opening 105 and the second side blade edge 103 are thus combined to form a “concave” shape.

In the embodiment, by providing the opening 105 on the inner cutting probe 100, there is no condition happened that the first side cutting and sucking port 202 of the outer probe 200 is blocked during the forward and backward cutting process of the inner probe 100 so as to avoid affecting the suction rate. This is because the suction of the vitreous body or tissue is completed by enabling the vitreous body or tissue entering the suction channel 101 via the first side cutting and sucking port 202 to be removed therefrom. If there is no opening 105 exists, the cutting and sucking port 202 is closed at the same time when the inner probe 100 moves forwardly to perform the cutting action, resulting in reduced suction efficiency. Therefore, the suction efficiency is greatly improved by providing the opening 105.

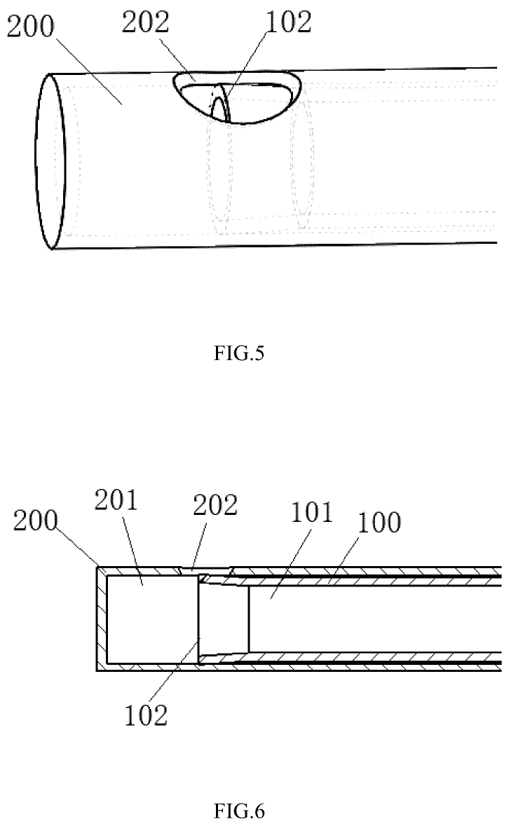

Alternatively, as shown with reference to FIGS. 5 and 6, another embodiment of the present invention provides a probe device comprising an inner cutting probe or tube 100 and an outer cutting probe or tube 200, wherein the outer cutting probe 200 has a tube shape with a sliding channel 201 along a tube longitudinal axis thereof, and the outer cutting probe 200 has an outer sidewall with a first side cutting and sucking port 202 serving to suck the vitreous body or tissue. The side cutting and sucking port 202 is in communicates with its sliding channel 201, and is located close to a front end of the outer cutting probe 200. The inner cutting probe 100 is configured to penetrate through the sliding channel 201 of the outer cutting probe 200 so that the inner cutting probe 100 can move within or be slidable with respect to the outer cutting probe 200. The inner cutting probe 100 also has a tube shape with a suction channel 101 along its tube longitudinal axis thereof, the inner cutting probe 100 has a front end with an end surface defining a first end blade edge 102 in communication with its suction channel 101. The first end blade edge 102 is a peripheral edge of an expanded cylinder, which is shaped as a trumpet. That is to say, the outer diameter of the expanded cylinder gradually increases in a forward direction from a right portion of the inner cutting probe towards a left portion of the inner cutting probe, with the outer diameter of the left portion of the expanded cylinder being greater than the outer diameter of the right portion of the expanded cylinder, and thus the outer side wall of the expanded cylinder at least partially engages with the inner side wall of the sliding channel 201 of the outer cutting probe 200. As shown in FIGS. 5 and 6, there is none of the second side blade edge 103 and the third side blade edge 104 exist on the inner cutting probe 100. It should be noted that in some embodiments, as the first end blade edge 102 is a peripheral edge of a front port of an expanded cylinder, the inner cutting probe 100 may or may not be provided with the second side blade edge 103 and the third side blade edge 104.

Note that the fitting clearance between the inner cutting probe 100 and the outer cutting probe 200 becomes one of the key indexes. It is necessary to ensure that the fitting clearance between the inner cutting probe 100 and the outer cutting probe 200 at the cutting position is as small as possible, otherwise, the cutting force on the vitreous body due to the relative movement between the inner cutting probe 100 and the outer cutting probe 200 disappears, turns into the compression on the vitreous body, and loses the effect of breaking the vitreous body. At the same time, in order to easily plug the inner cutting probe 100 into the outer cutting probe 200, a gap between them needs to be gradually increased, otherwise it is impossible to fit together with each other due to the existing manufacturing tolerances. These two requirements are the relatively contradictory requirements for the design.

In the embodiment as shown in FIGS. 5 and 6, the first end blade edge 102 formed on the peripheral edge of a front port of the expanded cylinder can ensure that the inner cutting probe 100 cooperates with the outer cutting probe 200 to complete the cutting action, and at the same time, sufficient fitting clearance is reserved in the remaining non-cutting places for allowing the inner cutting probe 100 to be easily fitted into the outer cutting probe 200.

Specifically, a needle tube with an outer diameter slightly smaller than the inner diameter of the outer probe 200 is selected, and a trumpet is formed at the front end of the inner cutting probe 100 by expanding a front end of a nozzle so as to form the inner probe 100 having the expanded cylinder with its greatest outer diameter equal to the inner diameter of the sliding channel 201. In the assembly process, the top wall of the inner cutting probe 100 is held by a jig, and then is inserted into the outer probe 200 due to the top wall of the inner cutting probe 100 is elastically deformed. When the inner cutting probe 100 enters into the outer cutting probe 200 at the position where the side cutting and sucking port 202 is located, the inner cutting probe 100 can tightly engage with the outer cutting probe 200, thereby forming a shear during the movement. Due to the high hardness and poor toughness of a stainless-steel tube, the internal probe is made from nitinol, which not only ensures the requirements of elasticity in assembly, but also ensures the strength and stiffness of the internal probe itself.

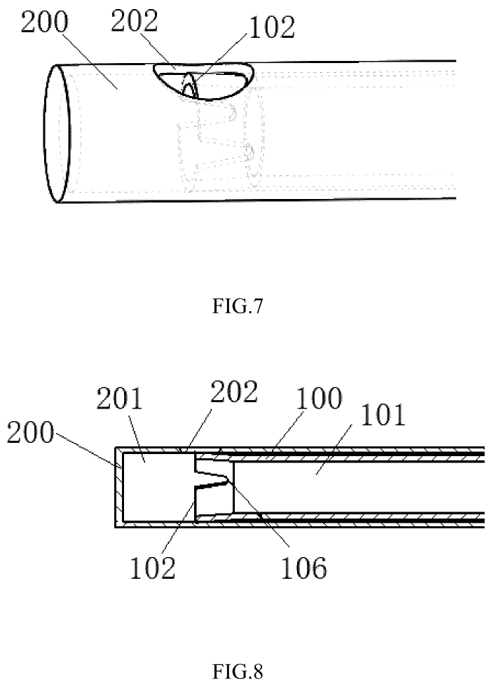

Further, as shown in FIGS. 7 and 8, based on the expanded cylinder of the inner cutting probe 100, one or more cutting grooves 106 are provided on a peripheral edge of the front port of the expanded cylinder and configured to extend from a left portion of the inner cutting probe towards a right portion of the inner cutting probe, and the wall or walls of one or more cutting grooves 106 tightly engage with the inner side wall of the sliding channel 201 of the outer cutting probe 200. The outer diameter of a front end of the expanded cylinder with one or more cutting grooves 106 in FIGS. 7 and 8 may be the same as or slightly greater than the inner diameter of a corresponding end of the sliding channel 201 of the outer cutting probe 200 to further ensure the cutting effect. The one or more cutting grooves 106 can ensure that there is spatial deformation of the inner cutting probe exists when the inner probe 100 is compressed during assembly, thereby reducing the limits for the elasticity of the material itself and for the size of the outer diameter of the first end blade edge 102. Herein, the cutting groove 106 has a certain bevel and fillet so that it can be helpful to reduce the cracking of the inner cutting probe 100 caused by stress concentration of the compression deformation during the assembly process.

The above-mentioned description is merely examples for the embodiments of the present application, and the scope of the embodiments of the present application is not limited thereto. Any changes or substitutions within the technical scope disclosed in the embodiments of the present application should be covered by the present application. Therefore, the protection scope of the embodiments of the present application should be determined by the appended claims.

What is claimed is:

1. A probe device comprising:
an inner cutting probe;
an outer cutting probe;
wherein the outer cutting probe has a first tube shape with a sliding channel along a first tube longitudinal axis thereof, and the outer cutting probe has an outer side wall with a first side cutting and sucking port in communication with the sliding channel, wherein the first side cutting and sucking port is configured to suck a vitreous body;
the inner cutting probe with a second tube shape is configured to penetrate through the sliding channel of the outer cutting probe and be slidable with respect to the outer cutting probe, the inner cutting probe having a suction channel along a second tube longitudinal axis thereof, the inner probe has a front end with an end surface defining a first end blade edge in communication with the suction channel of the inner cutting probe, an outer side wall of the inner probe is shaped to have a second side port with a top port edge, a bottom port edge, a second side blade edge and a third side blade edge opposite to the second side blade edge in a cross-sectional view thereof, a length of the top port edge is smaller than a length of the bottom port edge, the second side blade edge is located closer to the first end blade edge than the third side blade edge;
wherein the inner cutting probe is configured to have a left wall edge and a right wall edge, the top port edge is defined between the left wall edge and the right wall edge, a first cutting blade tip is formed, with a first cutting blade tip angle between the first end blade edge and the left wall edge ranging from 40 degrees to 60 degrees; a second cutting blade tip is formed, with a second cutting blade tip angle between the third side blade edge and the right wall edge ranging from 40 degrees to 60 degrees; and a third cutting blade tip is formed, with a third cutting blade tip angle between the second side blade edge and the left wall edge ranging from 40 degrees and 60 degrees;
when the inner cutting probe is slidable within the sliding channel of the outer cutting probe in a forward direction, the first end blade edge is to perform a first cutting by the first cutting blade tip piercing into the vitreous body of the outer cutting probe along the forward direction, and the inner cutting probe continues to move forwardly such that the third side blade edge is to perform a second cutting by the second cutting blade tip piercing into the vitreous body of the outer cutting probe along the forward direction;
when the inner cutting probe is slidable within the sliding channel of the outer cutting probe in a backward direction, merely the second side blade edge is to perform a third cutting by the third cutting blade tip piercing into the vitreous body of the outer cutting probe along the backward direction.

2. The probe device according to claim 1, wherein an opening is formed between second side blade edge and the third the third side blade edge.

3. The probe device according to claim 1, wherein the first end blade edge is formed on a peripheral edge of a front port of an expanded cylinder, an outer diameter of the expanded cylinder gradually increases in a forward direction from the a right portion of the inner cutting probe towards a left portion of the inner cutting probe, and an outer side wall of the expanded cylinder at least partially engages with the inner side wall of the sliding channel of the outer cutting probe.

4. The probe device according to claim 3, wherein one or more cutting grooves are provided on a peripheral edge of the front port of the expanded cylinder and configured to extend from the left portion of the inner cutting probe towards the right portion of the inner cutting probe, and the outer diameter of a front end of the expanded cylinder with one or more cutting grooves is greater than or equal to the inner diameter of a corresponding end of the sliding channel of the outer cutting probe; and
the expanded cylinder can penetrate through the sliding channel when the expanded cylinder is deformed by applying a force thereto.
